(19) United States
(12) Patent Application Publication
(10) Pub. No.: US 2025/0057527 A1
(43) Pub. Date: Feb. 20, 2025

(54) SUTURING NEEDLES AND RELATED SYSTEMS AND METHODS

(71) Applicant: Intuitive Surgical Operations, Inc., Sunnyvale, CA (US)

(72) Inventor: Bruce M. Schena, Sunnyvale, CA (US)

(21) Appl. No.: 18/808,518

(22) Filed: Aug. 19, 2024

Related U.S. Application Data
(60) Provisional application No. 63/533,248, filed on Aug. 17, 2023.

(51) Int. Cl. A61B 17/06; A61B 17/00

(52) U.S. Cl. CPC A61B 17/06066; A61B 2017/00477; A61B 2017/00862; A61B 2017/06095; A61B 2017/061

(57) ABSTRACT

Implementations of suturing needles, suturing devices, and related methods are provided. An illustrative suturing needle includes a needle body having a leading end and a trailing end. The needle body includes an outer tubular body having a proximal end and a distal end configured to pierce tissue, and at least one at least one elongate inner body made of superelastic material disposed within and operably coupled to the outer tubular body. The outer tubular body deforms from a first configuration into a second configuration in response to the application of force by the at least one elongate inner body on the outer tubular body.

### CROSS-REFERENCE TO RELATED APPLICATION

The present patent application claims the benefit of priority to U.S. Provisional Patent Application No. 63/533,248, filed Aug. 17, 2023. The aforementioned patent application is incorporated by reference herein in its entirety for all purposes.

### TECHNICAL FIELD

Aspects of the present disclosure relate to devices, systems, and methods for performing a suturing procedure. For example, aspects of the present disclosure relate to needles for suturing devices in support of remote surgical, diagnostic, therapeutic, and other treatment procedures.

### INTRODUCTION

Sutures are used in a variety of surgical and other applications, such as closing ruptured or incised tissue, soft tissue attachment, attachment of grafts, etc. Additionally, sutures may have other medical and/or non-medical uses. Conventionally, suturing is accomplished by penetrating tissue with the sharpened tip of a suturing needle that has a thread of suturing material attached to the opposite blunt end of the needle. The needle is then pulled through the tissue, causing the attached thread of suturing material to follow the path of the needle. Typically, a knot is tied at the trailing end of the thread to anchor the first stitch. This action is performed repetitively with application of tension to the needle to pull a length of the thread through the tissue using subsequent stitches until the tissue is sutured as desired with one or more stitches.

While the above-described suturing process can be performed manually, automated suturing systems also exist. Such systems can include a needle driver device that has an open, C-shaped portion into which tissue segments are introduced. The C-shaped portion defines two arms, each with an entry/exit point for a curved needle. The curved needle is driven around a track (generally following the C-shaped portion) and across the opening in the C-shaped portion to draw a thread of suturing material into the needle driver device through the tissue segments, similar to the manual suturing process discussed above. It is desirable to provide improved suturing needles that are easier to use to facilitate such suturing procedures. The present disclosure addresses these, and other issues in the art, as set forth below.

### SUMMARY

Example embodiments of the present disclosure may solve one or more of the above-mentioned problems and/or may demonstrate one or more of the above-mentioned desirable features. Other features and/or advantages may become apparent from the description that follows.

In accordance with some aspects of the present disclosure, a suturing needle is provided. The suturing needle includes a needle body having a leading end and a trailing end. The needle body includes an outer tubular body having a proximal end and a distal end configured to pierce tissue, and at least one at least one elongate inner body made of superelastic material disposed within and operably coupled to the outer tubular body. The outer tubular body deforms from a first configuration (typically when in a constrained condition) into a second configuration (typically when no longer constrained) in response to the application of force by the at least one elongate inner body on the outer tubular body.

In some implementations, the outer tubular body can define at least one opening therethrough when the outer tubular body is in the first configuration that decreases in spatial extent when the outer tubular body deforms from the first configuration into the second configuration. In some implementations, the at least one opening can define a plurality of openings defined along a length of the outer tubular body. In accordance with some aspects, the first configuration of the outer tubular body can be a generally linear configuration. The second configuration of the outer tubular body can be a curved configuration, such as an arcuate configuration, a splined shape, or an out of plane shape. The at least one opening can be formed, for example, by a laser ablation process or an etching process.

In further accordance with the disclosure, each opening in the plurality of openings can be defined along a first lateral side of the outer tubular body. Each opening in the plurality of openings can be defined along a pathway that is parallel to a central longitudinal axis of the outer tubular body.

In some implementations, each opening in the plurality of openings can be defined along a pathway that is curved about a central longitudinal axis of the outer tubular body along a length of the outer tubular body, such that it wraps about the outer tubular body helically. In some implementations, the at least one opening in the plurality of openings can have a perimeter including interlocking features on opposing sides of the at least one opening that interlock when the suturing needle is in the second configuration to resist torsional forces applied to the outer tubular body.

In some implementations of the suturing needle, no openings are defined along a second lateral side of the outer tubular body opposite the first lateral side of the outer tubular body, wherein the first lateral side of the tubular body defines one or more openings therethrough. The second lateral side of the outer tubular body opposite the first lateral side of the outer tubular body can be defined by a continuous surface free from openings, or free from openings that affect the deformation from the outer tubular body from the first configuration into the second configuration. In accordance with further implementations, the outer tubular body is configured to not be diametrically expandable and/or diametrically contractable.

In some implementations, the suturing needle can be configured such that a cross-sectional height of the outer tubular body does not change when the outer tubular body deforms from the first configuration into the second configuration. In further implementations, the outer tubular body has sufficient column strength when in the second configuration to be advanced through tissue to suture the tissue without deformation of the suturing needle. For example, the outer tubular body can be generally toroidally shaped when in the second configuration. The outer tubular body can be configured to resist deformation when advanced through tissue in the second configuration during a suturing operation. In some implementations, the outer tubular body is rigid when in the second configuration to be advanced through tissue to suture the tissue without deformation of the shape of the suturing needle.

In some implementations, the outer tubular body can define at least one opening on a second lateral side of the outer tubular body opposite the first lateral side of the outer tubular body that increases in spatial extent when the outer tubular body deforms from the first configuration into the second configuration. For example, the at least one opening on the second lateral side of the outer tubular body can be configured to be engaged by a needle advancement drive of a suturing device when the outer tubular body is in the second configuration. In such implementations, the suturing needle is preferably sufficiently rigid to be advanced through tissue when in the second configuration.

The components of the needle can be joined in a variety of manners. For example, in some implementations, a first end region of the at least one elongate inner body can be fixedly coupled to a leading end region of the outer tubular body and a second end region of the at least one elongate inner body can be fixedly coupled to a trailing end region of the outer tubular body. Attachment may be accomplished by one or more of welding, brazing, soldering, adhesive(s), crimping, and the like.

In some implementations, the at least one elongate inner body can be located proximate a geometric centerline of the outer tubular body. In further implementations, the at least one elongate inner body can be displaced from a geometric centerline of the outer tubular body. For example, the at least one elongate inner body can be displaced from a geometric centerline of the outer tubular body toward the at least one opening. In accordance with further aspects of the disclosure, the outer tubular body can have a cross sectional shape selected from a circle, a rectangle, a triangle, a trapezoid, and a splined shape.

In accordance with still further aspects of the disclosure, the outer tubular body of the suturing needle can be formed from a plurality of adjacent segments. Each said segment can define a lumen therethrough to receive the at least one elongate inner body therethrough. For example, a plurality of said adjacent segments can define at least one rounded end to facilitate articulation against an adjacent segment. The at least one rounded end can be generally spherical in shape, or may be defined by a curved plane.

In accordance with further aspects of the disclosure, when the outer tubular body is in the second configuration, the at least one elongate inner body can be at least in a partial state of phase change to maintain rigidity of the elongate outer body in the second configuration. In some implementations, the superelastic material can include an alloy of nickel and titanium. In accordance with further aspects, the suturing needle can include a plurality of elongate inner bodies, wherein each said elongate inner body is formed at least in part from superelastic material.

The suturing needle can be mechanically flexible when the outer tubular body is in the first configuration, and the suturing needle can be mechanically rigid when the outer tubular body is in the second configuration. This can facilitate delivery of the needle through an elongate passageway of an endoscope, for example.

In further accordance with the disclosure, a needle delivery catheter is provided that includes an elongate main body portion, a suturing needle as described herein operably coupled thereto, and a sheath operably coupled to a distal region of the elongate main body portion. The sheath can define a lumen along at least a portion of its length. The lumen can be configured to removably receive the suturing needle therein.

In some implementations, the lumen can be configured to surround the leading end of the suturing needle. In some embodiments, the suturing needle can be removed from the lumen by moving the elongate main body portion with respect to the sheath. If desired, the sheath can be configured to resist the suturing needle from deforming from the first configuration into the second configuration. In further implementations, the needle delivery catheter can further include a removable restraining wire disposed within the outer tubular body of the suturing needle to prevent the suturing needle from deforming from the first configuration into the second configuration. The restraining wire can be withdrawn proximally to permit the outer tubular body to deform from the first configuration into the second configuration. It will be further appreciated that the elongate main body portion of the needle delivery catheter can define an opening at a distal end thereof to receive a proximal end of the suturing needle. The elongate main body portion of the needle delivery catheter can define a lumen along at least a portion of its length to receive a trailing suture therethrough, as well as a restraining wire, if desired.

In still further accordance with the disclosure, a needle delivery catheter is provided that includes an elongate main body portion, a suturing needle as described herein operably coupled thereto, and a removable restraining wire disposed within the outer tubular body of the suturing needle to prevent the suturing needle from deforming from the first configuration into the second configuration. If desired, the needle delivery catheter can further include a covering to cover a free end of the needle.

In further accordance with the disclosure a suturing device is provided that includes a housing defining a needle track therein to slidably receive a suturing needle therein, a drive to advance the suturing needle along the needle track and through a tissue segment. The drive can be configured to engage and disengage from the suturing needle to advance the needle through the tissue segment. The suturing device can further include a needle delivery catheter as described herein, as well as a tissue capture catheter to grasp and manipulate tissue.

In further accordance with the disclosure, the needle track can be an arcuate needle track having a first end and a second end. The housing can further define a gap between the first end and second end of the arcuate needle track, wherein the gap is configured to receive the tissue segment therein to be sutured. A suturing needle can be utilized that is configured to deform into an arcuate shape when the outer tubular member of the suturing needle in the second configuration. The drive can be configured to releasably engage the suturing needle and advance the arcuate needle along the arcuate needle track and across the gap from a starting position to an ending position during a drive stroke. In some implementations, the needle delivery catheter and the drive can cooperate to engage the suturing needle with the needle track.

In further accordance with the disclosure, methods of suturing tissue are provided. In accordance with some aspects, the method can include providing a suturing device as described herein, advancing the needle delivery catheter along a passageway of the suturing device toward the needle track, disposing the suturing needle in the needle track and engaging the drive with the needle, and advancing a distal end portion of the suturing device proximate tissue to be sutured, wherein the suturing needle includes a first length of suturing material attached thereto. The method can further include advancing the suturing needle having along the needle track through the tissue a plurality of times to form a plurality of stitches.

In accordance with further aspects, the method can further include engaging the needle delivery catheter with the suturing needle after the stitches have been formed, and removing the needle delivery catheter and needle from the suturing device. The method can still further include directing a tissue capture catheter into tissue to be sutured, and retracting the tissue into a pathway of the needle prior to suturing.

In further accordance with the disclosure, implementations of a suturing needle are provided having a needle body with a leading end and a trailing end, the needle body comprising a first body having a proximal end and a distal end configured to pierce tissue, and at least one at least one elongate body made of superelastic material operably coupled to the first body, wherein the first body deforms from a first configuration into a second configuration in response to the application of force by the at least one elongate body on the first body.

In some implementations, the least one elongate body can be disposed parallel to the first body. The least one elongate body can be operably coupled to the first body at a plurality of locations along a length of the first body.

Additional objects, features, and/or advantages will be set forth in part in the description which follows, and in part will be obvious from the description, or may be learned by practice of the present disclosure and/or claims. At least some of these objects and advantages may be realized and attained by the elements and combinations particularly pointed out in the appended claims.

It is to be understood that both the foregoing general description and the following detailed description are illustrative and explanatory only and are not restrictive of the claims; rather the claims should be entitled to their full breadth of scope, including equivalents.

### BRIEF DESCRIPTION OF THE DRAWINGS

The present disclosure can be understood from the following detailed description, either alone or together with the accompanying drawings. The drawings are included to provide a further understanding of the present disclosure and are incorporated in and constitute a part of this specification. The drawings illustrate one or more embodiments of the present teachings and together with the description explain certain principles and operation. In the drawings:

FIG. 1 depicts an isometric view of an illustrative suturing needle in accordance with some embodiments of the present disclosure in a straightened configuration.

FIG. 2 depicts a cross sectional view of the suturing needle of FIG. 1.

FIG. 3 is a top isometric view of the suturing needle of FIG. 1 in a curved configuration.

FIG. 4 depicts a cross section of the needle as depicted in FIG. 3.

FIGS. 5A and 5B are schematic views of a further illustrative suturing needle in accordance with some embodiments of the present disclosure in a straightened configuration and a curved configuration, respectively.

FIG. 6A is a schematic view illustrating interlocking features that can be formed into the edges of openings formed into a needle in accordance with the present disclosure.

FIG. 6B is a schematic view showing varied placement of openings through an outer tubular wall of the suturing needle in further accordance with the disclosure.

FIG. 7 is a schematic view of a needle delivery catheter in accordance with the present disclosure.

FIG. 8 is a schematic view of a suturing system in accordance with the present disclosure.

FIG. 9 is a schematic diagram for a robotically-assisted manipulator system, according to some examples.

FIG. 10A is a schematic diagram of an instrument system according to examples described herein.

FIG. 10B illustrates a distal portion of the instrument system of FIG. 10A with an extended example of an instrument according to examples described herein.

### DETAILED DESCRIPTION

The present disclosure provides various embodiments of suturing needles and associated suturing devices and methods. Suturing needles according to various embodiments of the present disclosure include features that facilitate suturing operations, particularly in endoscopic procedures.

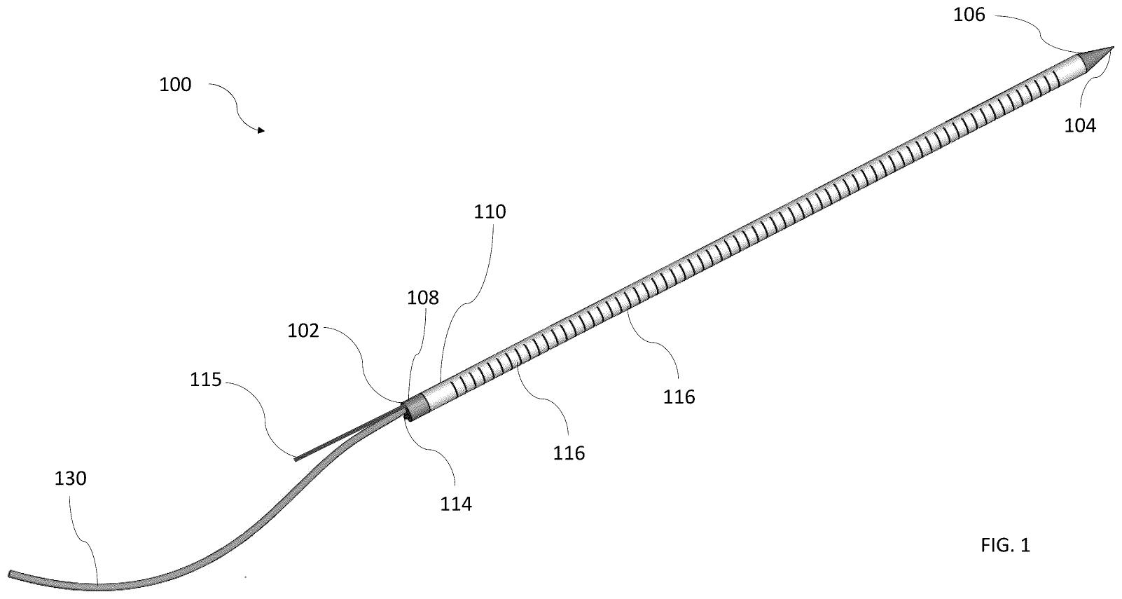

In accordance with some aspects of the present disclosure, a suturing needle is provided. For purposes of illustration, and not limitation, a first representative implementation of a suturing needle is presented in FIGS. 1-4.

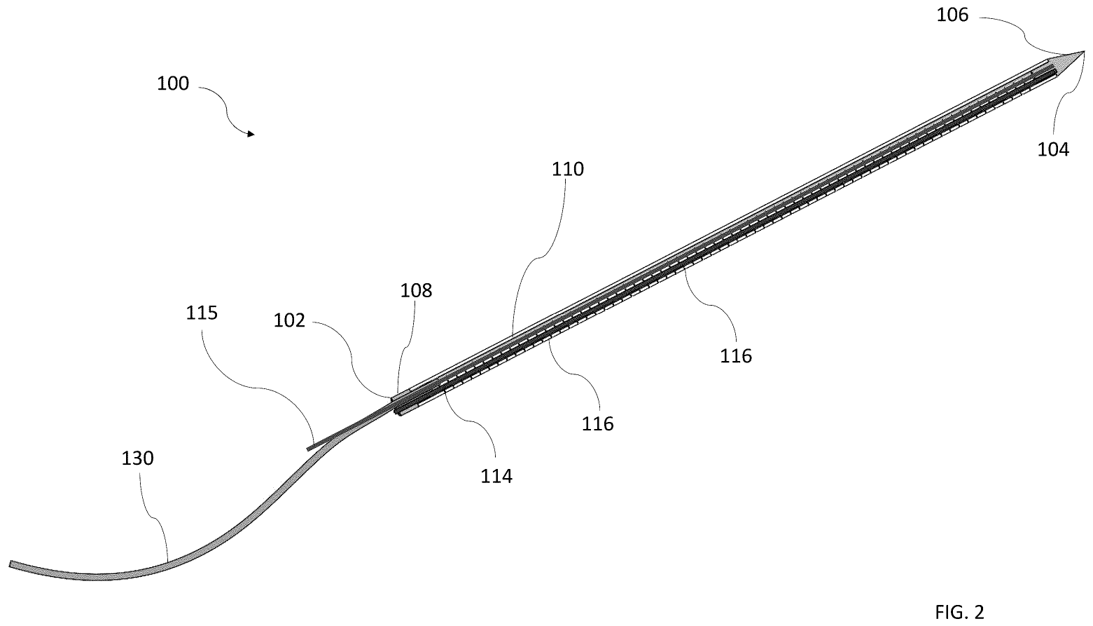

As depicted in FIGS. 1-2, a suturing needle 100 is provided that includes a needle body having a leading end terminating in a point, or tip configured to pierce tissue 104 and a trailing end 102. The needle 100 depicted in FIGS. 1-4 is formed from an outer tubular body 110 having a proximal end and a distal end. The distal end of the outer tubular body 110 receives a needle tip portion 106 therein, and the proximal end 102 receives a plug or retainer 108 that can be configured to engage a distal end of a length of suturing material 130.

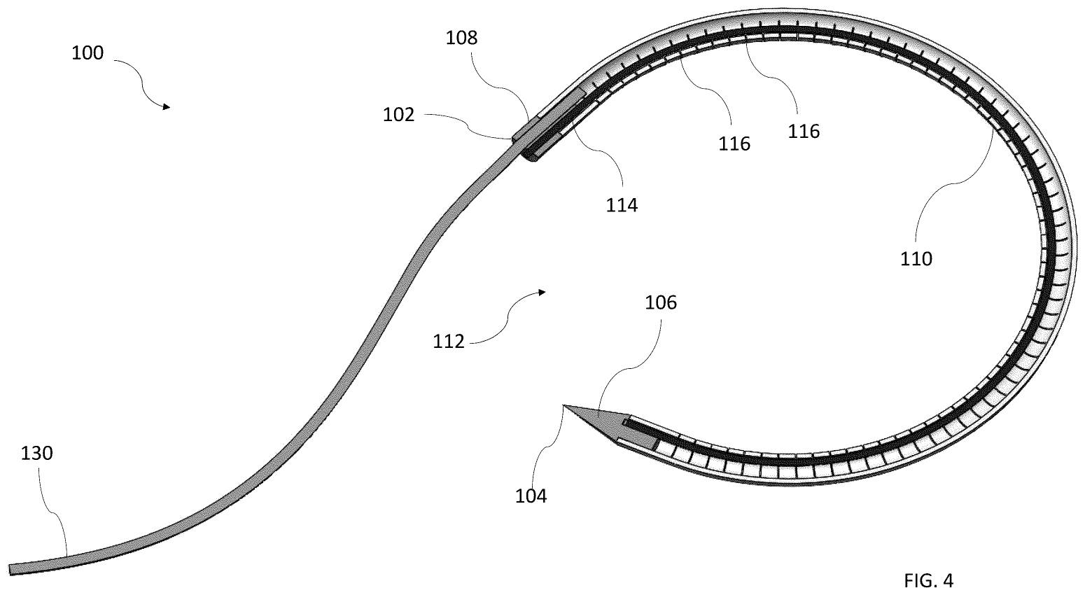

As further depicted in FIGS. 1-4, the needle 100 can include one or more elongate inner bodies 114 made from a superelastic material, such as an alloy of nickel and titanium. The ratio of nickel and titanium may be selected, for example, to enhance the superelastic attributes of the alloy in contrast to the shape memory aspects of the alloy. The elongate inner body 114 is preferably rigidly coupled to a location proximate each end of the outer tubular body 110 by way of welding, brazing, soldering, crimping, or the like. As depicted in FIGS. 2 and 4, the distal end of the elongate inner body 114 is anchored into material of the needle tip 106, and the proximal end of the elongate inner body 114 is anchored into material of plug 108. The outer tubular body 110 can have a cross sectional shape selected from a circle, an oval, a rectangle, a triangle, a trapezoid, and a splined shape, among others.

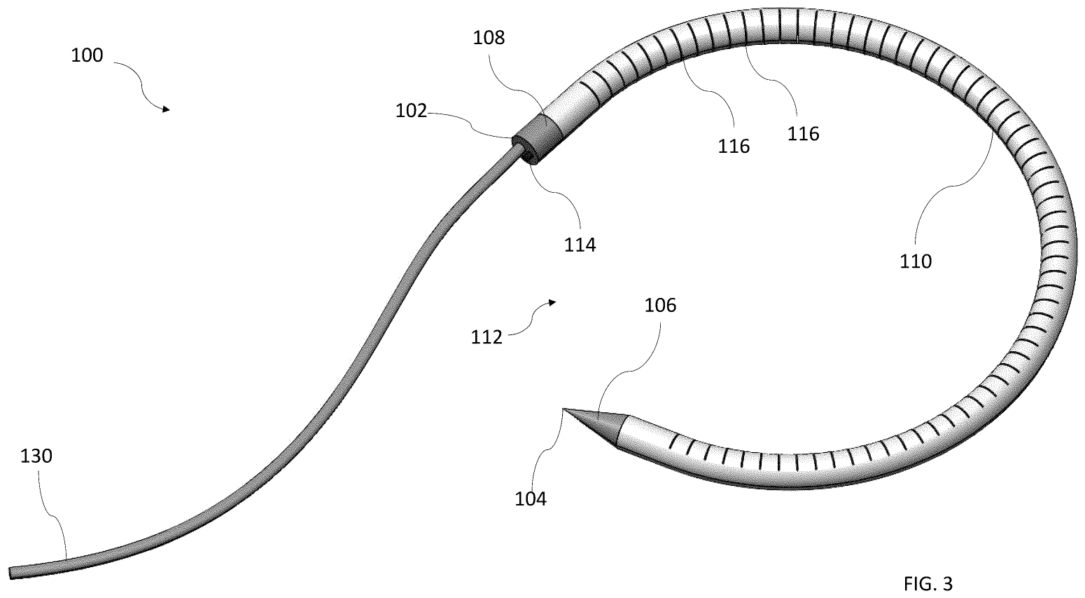

With continuing reference to FIGS. 1-4, the outer tubular body 110 of the needle 100 defines a plurality of openings 116 therethrough. As depicted, the openings 116 are depicted as “cuts” across the transverse width of the outer tubular member 110. Openings 116 can be formed by etching processes or laser ablation performed on a hypotube-type material. The openings 116 are shaped and arranged to “collapse” on themselves when tension is applied by the superelastic elongate inner body 114 that is in an elongated, stretched state when the needle is in a straightened configuration. When the outer tubular body 110 is unconstrained, the outer tubular body 110 can contract from this first, straightened configuration and form a curved shape, wherein the openings 116 decrease in spatial extent, to form a second, curved configuration as depicted in FIGS. 3 and 4.

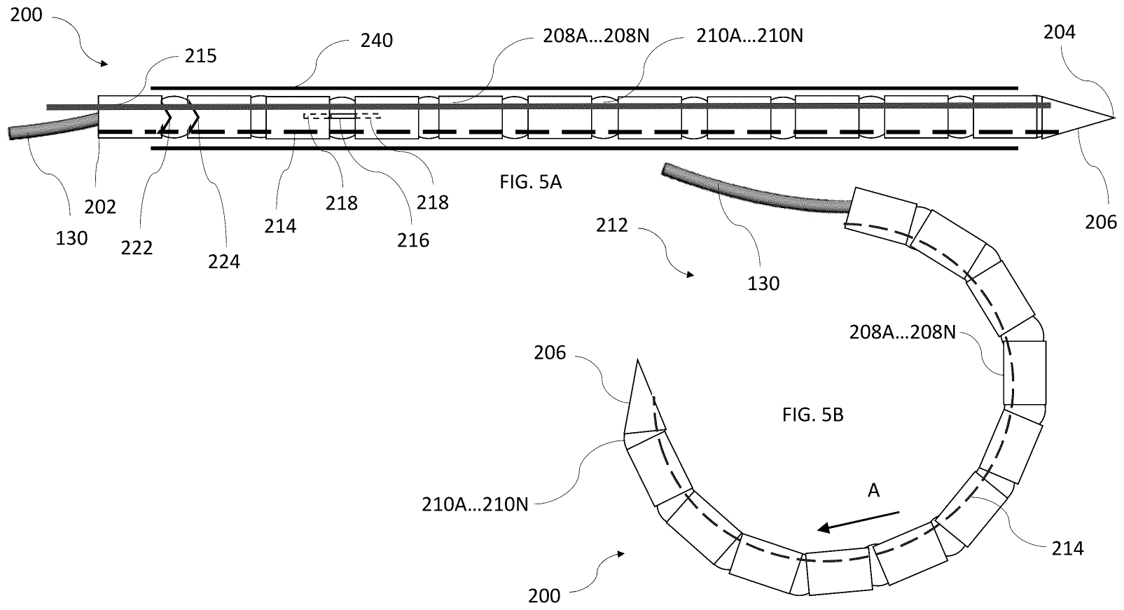

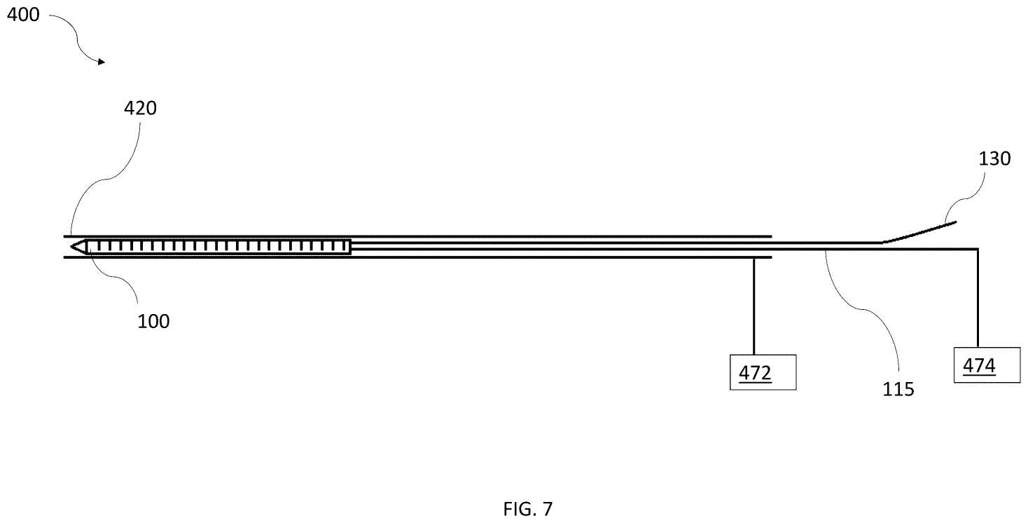

The elongate body of the needle 100 is configured to be adjusted into a straightened configuration as depicted in FIGS. 1-2 by stretching the elongate inner body 114. In the straightened configuration, the needle 100 can be configured to have sufficient flexibility to be advanced through a delivery lumen to a needle track of a suturing device (e.g., 500), discussed in further detail below. To facilitate maintaining the needle in a generally straight configuration for delivery, a variety of approaches are available. In some implementations, as depicted in FIGS. 1-2, a stabilizing rod 115, such as a relatively stiff wire, can be slidably received within the lumen of the outer tubular member 110. The stabilizing rod 115 can be any suitable material for imparting stiffness to the needle and to resist the bending force applied by the elongate inner body 114. A delivery sleeve or an outer tubular member (e.g., 240 in FIG. 5A or 420 in FIG. 7 can additionally or alternatively be used to maintain needle 100 in the relatively straight, delivery configuration.

It will be appreciated by those of skill in the art that the first shape configuration used to deliver the needle 100 and the second configuration of the needle 100 when unconstrained can be varied. For example, while the first configuration can be relatively straight, if needed, the needle 100 can be configured to provide a desired curvature to facilitate delivery, depending on the application, or may even include an out of plane shape, such as a helical or a splined shape. The second configuration of the outer tubular body 110, and needle 100, can be a curved configuration, such as an arcuate configuration, a splined shape, or an out of plane shape such as a helical shape, three-dimensional splined shape, and the like. When in the second configuration, a gap 112 can be defined between the proximal end 102 of the needle 100 and the pointed distal end 104 of the needle 100.

As depicted in FIGS. 1-4, no openings are defined along a second lateral side of the outer tubular body opposite the first lateral side of the outer tubular body. This facilitates collapse of the body into a curved shape when in the second configuration, wherein the second lateral side of the outer tubular body becomes an outer radial surface of the needle 100. At the same time, having a radially outer surface as depicted provides structural rigidity and pushability of the suturing needle 100 when in the second configuration. As such, the second lateral side of the outer tubular body opposite the first lateral side of the outer tubular body can be defined by a continuous surface free from openings, or free from openings that affect the deformation from the outer tubular body from the first configuration into the second configuration. The illustrated implementation depicts an outer tubular body that is bendable due to the presence of the openings 116, and wherein the outer tubular body is not diametrically expandable and/or diametrically contractable, in contrast to a stent-like structure.

In the depicted implementation 100, it will be appreciated that a cross-sectional height of the outer tubular body of the needle does not change when the outer tubular body deforms from the first configuration into the second configuration. Reference to the “height” in this aspect refers to the height of the needle of FIG. 4 resting on a flat surface, which in this implementation corresponds to the diameter of the outer tubular member. It will be appreciated that the outer tubular body has sufficient column strength, at least when in the second configuration, to be advanced through tissue to suture the tissue without deformation of the suturing needle. The outer tubular body can thus be configured to resist deformation when advanced through tissue in the second configuration during a suturing procedure. In some implementations, the outer tubular body is preferably rigid when in the second configuration to be advanced through tissue to suture the tissue without deformation of the shape of the suturing needle.

In some implementations, the outer tubular body can define at least one opening on a second lateral side of the outer tubular body opposite the first lateral side of the outer tubular body that increases in spatial extent when the outer tubular body deforms from the first configuration into the second configuration. For example, the at least one opening on the second lateral side of the outer tubular body can be configured to be engaged by a needle advancement drive of a suturing device when the outer tubular body is in the second configuration. In such implementations, the suturing needle is preferably sufficiently rigid to be advanced through tissue when in the second configuration.

In accordance with still further aspects of the disclosure, the outer tubular body of the suturing needle can be formed from a plurality of adjacent segments. Each said segment can define a lumen therethrough to receive the at least one elongate inner body therethrough. For example, a plurality of said adjacent segments can define at least one rounded end to facilitate articulation against an adjacent segment. The at least one rounded end can be generally spherical in shape, or may be defined by a curved plane.

FIGS. 5A and 5B present schematic views of a further implementation of a needle 200 in accordance with the present disclosure. Needle 200 includes a proximal end 202, a distal end 204, and a needle tip segment 206. A difference between needle 100 and needle 200 is that the continuous outer tubular body 110 of needle 100 has been replaced with a segmented outer tubular body made from segments 208 and 210 that permit relative articulating movement between the segments. The segments are illustrated as being rectangular tubular segments 208A-208N that interfit with rounded segments 210A-210N that define passageways therethrough to define a main passageway along the interior of the needle 200. It will be appreciated that the segments can be varied in shape and size. For example, a plurality of identical or substantially identical segments can be used that have a rounded convex surface at one end, and a rounded concave surface at the other end, wherein the rounded convex surface of a first segment is received within the rounded concave surface of a second segment, which pattern can be repeated along the length of the needle 200. Any suitable arrangement may be used that provides relative articulation to permit the needle to curve to a desired geometry.

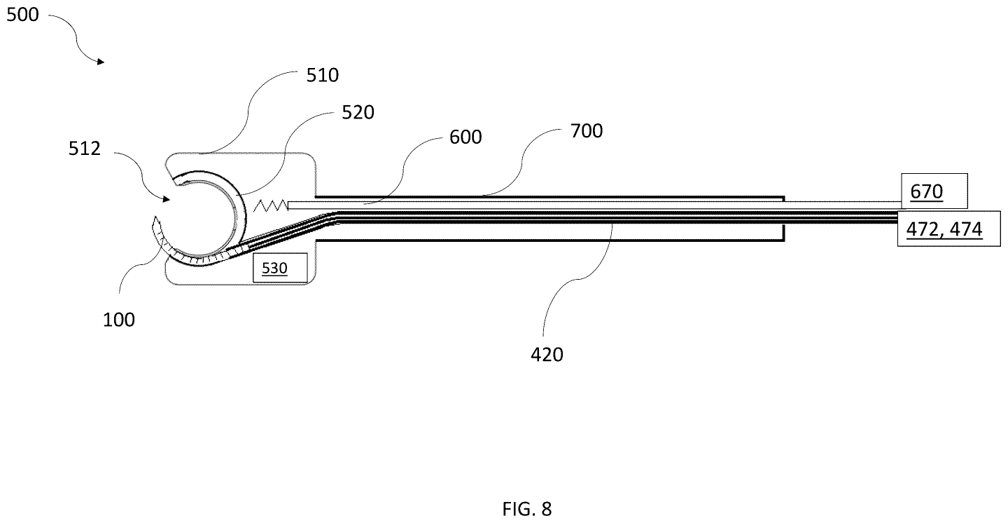

With continuing reference to FIGS. 5A and 5B, as with embodiment 100, needle 200 can include one or more lengths 214 of superelastic material along its length that cause the needle to curve into a desired shape when the needle 200 is unconstrained to permit the superelastic material 214 to contract axially. Constraint can be provided by a removable stiffening wire or other elongate rod 215 that is received within a longitudinal passage of the needle 200, which may be anchored within a tip portion 206 of the needle 200, as with implementation 100. Moreover, a tubular sheath 240 can also be slidably disposed about the needle 200, and if desired, can surround a tip of the needle (e.g., 100, 200) when delivering the needle (e.g., 100, 200) to the needle track 520 of a suturing device 500 (see FIGS. 7-8). A length of suturing material 130 preferably trails from the proximal end 200 of the needle as with implementation 100. FIG. 5A depicts needle 200 in a straightened configuration within delivery sheath 240 and/or with stiffening rod 215, whereas FIG. 5B depicts needle 200 after lateral restraint is removed, permitting the needle 200 to curve around into its second configuration, wherein a gap 212 is formed between the trailing and 202 of the needle and the pointed end 204 of the needle 200.

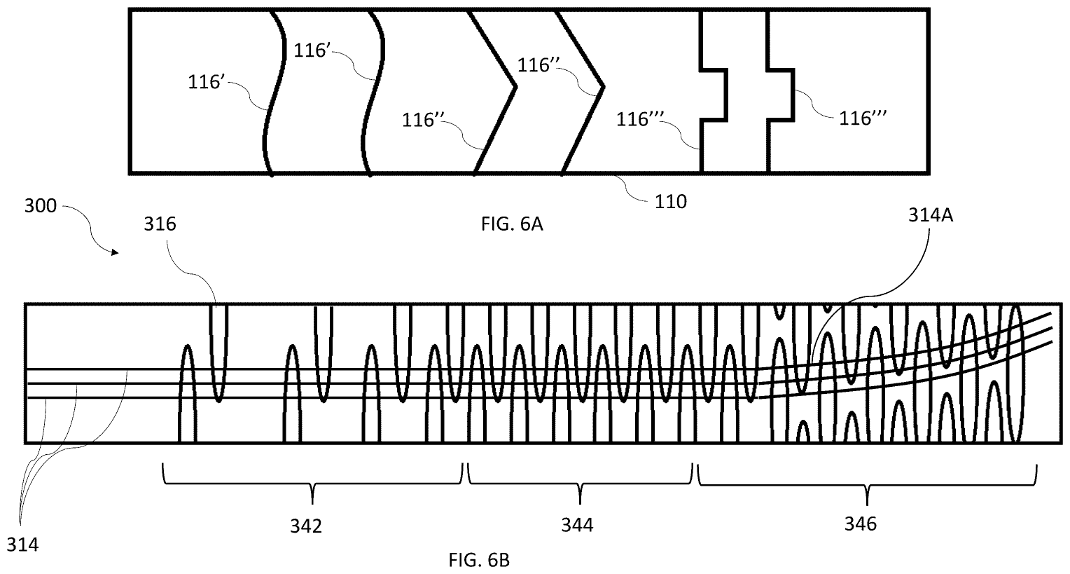

If desired, needles in accordance with the present disclosure can be provided with alignment features on opposing sides of each opening 116 to cause the two sides of the opening to mechanically engage to resist application of torsional loads applied about a central axis of the needle 100, 200. For purposes of illustration only, FIG. 6A is a schematic view illustrating interlocking features that can be formed into the edges of openings (116′, 116″, 116′″) formed into a wall of a needle in accordance with the present disclosure. FIG. 6A presents a plan view of what a viewer would see when a needle has taken on a curved configuration such as needle 100 in FIGS. 3-4, wherein the needle wall is being viewed from inside of the curvature of the needle such that the openings 116 have closed on themselves. Any desired complementary pattern can be used so that the openings 116 close in a predetermined relationship. For example, openings 116′ are configured to close in a sinusoidal shape across a transverse width of the outer tubular body 110. Openings 116″ are configured to close in a sawtooth shape across a transverse width of the outer tubular body 110, while openings 116′″ are configured to close in a keyed shape across a transverse width of the outer tubular body 110. With continuing reference to FIG. 5A, needle 200 can similarly be provided with alignment features 222, 224 on an external surface of adjacent segments or nearby segments that lock together when the needle is in the second configuration. Additionally or alternatively, guide grooves 218 can be provided on an inner tubular surface of segments 208 to receive raised rails 216 formed into intermediate segments 210. This can provide alignment while the needle is closing from the first configuration to the second configuration.

It will be appreciated that the location, frequency, and shapes of the plurality of openings 116 can be used to impart a desired curvature to the outer tubular member 110 can be varied. In some implementations, such as in FIGS. 1-4, the openings 116 can defined along a first lateral side of the outer tubular body 110 that then becomes an inwardly facing toroidal surface of the needle 100 as depicted in FIGS. 3 and 4. In so doing, each opening 116 in the plurality of openings 116 can be defined along a pathway that is parallel to a central longitudinal axis of the outer tubular body.

With reference to FIG. 6B, in some implementations of a needle 300, each opening 316 in the plurality of openings can be defined along a pathway that is curved about a central longitudinal axis of the outer tubular body along a length of the outer tubular body as illustrated in region 346, such that the openings wrap about the outer tubular body helically. As further illustrated in FIG. 6B, openings 316 may be provided on opposing lateral faces of the needle body. This can move the point of bending closer to a middle of the tubular body of the needle rather than toward the outside face of the curved needle depicted in FIGS. 3-4.

Additionally, by providing openings in both faces of the needle, the openings can close together on the inner curved surface of the needle, and open wider on the radially outer surface of the needle, once curved. For example, widened openings on the radially outer face of the toroidal needle can, if desired, be configured to be engaged by a needle advancement drive of a suturing device or an anti-backup pawl or pin of the suturing device.

It will be further appreciated that the elongate superelastic body can be placed in various locations within the outer tubular body. For example, in some implementations, the elongate inner body (e.g., 114, 214, 314) can be located proximate a geometric centerline of the outer tubular body (e.g., 110). As illustrated in FIGS. 1-4, in contrast, the at least one elongate inner body 114 is displaced from a geometric centerline of the outer tubular body toward a lateral face of the tubular body 110 toward the openings 116. With continuing reference to FIG. 6B, more than one elongate body 314 is provided that can be located along a center of the body of needle 300, off the centerline, or along a helical pathway, as depicted in region 346. The openings 316 can be spaced uniformly along the length of the needle as in region 344, or non-uniformly as in region 342, such as to provide a splined curvature to the needle or other desired shape. Distributing the openings about the outer tubular body in a spiral fashion as in region 346 can help the needle to take on a three-dimensional shape when in the second configuration, and this can be further enhanced by placement of the superelastic segments 314 along and around the inner lumen of the outer tubular body of the needle 300.

In further accordance with the disclosure, implementations of a suturing needle are provided having a needle body with a leading end and a trailing end. The needle body can in turn include a first body having a proximal end and a distal end configured to pierce tissue, and at least one at least one elongate body made of superelastic material operably coupled to the first body. The first body can deform from a first configuration into a second configuration in response to the application of force by the at least one elongate body on the first body. While the first body can be an outer tubular member as described and the elongate body of superelastic material can be rod-shaped, it will be appreciated that the first body can be a solid member that is attached to the superelastic material at one or more locations. Alternatively, the superelastic material can be operably coupled to an outer portion of a tubular member to form the suturing needle.

The elongate inner body (e.g., 114, 214, 314) that is made from superelastic material may be configured such that it is always under some degree of tension. This can maintain tension on the needle (e.g., 100, 200) when in the second (e.g., curled) configuration to resist out of plane torque forces and to otherwise maintain alignment of the needle. The needle (e.g., 100, 200) can be provided with more than one such superelastic elongate element. Such elements can be located on or off the center axis of the needle in order to achieve a predetermined bending outcome when the needle is not restrained. In some implementations, the suturing needle (e.g., 100, 200) can be mechanically flexible when the outer tubular body is in the first configuration to facilitate delivery of the needle through an elongate passageway of an endoscope, for example.

In further accordance with the disclosure, a needle delivery catheter is provided to deliver a needle as described herein to a target location, such as a needle track of an endoscopic suturing device.

For purposes of illustration, and not limitation, as depicted in FIG. 7, a needle delivery catheter 400 is provided that that includes an elongate main body portion that may function as a sheath 420 to contain and restrain the needle (e.g., 100). The device can further include an elongate rod (e.g., 115) to push the needle through the tubular sheath 420. A length of suturing material 130 can be provided that trails from the proximal end (e.g., 102, 202) of the needle and passes through the lumen of sheath 420. The suture material 130 can be pulled either directly, or by way of a tensioning device or actuator (not shown) to pull the needle proximally within the sheath 420. The elongate rod 115 can be a stiffening rod that passes through the needle, or may be a pusher rod that engages a proximal end of the needle to push the needle distally. The elongate rod 115 may be operably coupled to an actuator or handle 474, and the sheath 420 may similarly be operably coupled to an actuator or handle 472 to push the sheath 420 distally or retract it proximally.

With continuing reference to FIG. 7, as depicted, the sheath 420 can be configured to surround the leading, or distal end (e.g., 104, 204) of the suturing needle (e.g., 100, 200). In the implementation of FIG. 7, the suturing needle (e.g., 100) can be removed from the lumen by moving the stabilizer/stiffening rod 115 respect to the sheath 420. If desired, the sheath can be sufficiently stiff to resist the suturing needle from deforming from the first configuration into the second configuration. If a rod or wire 115 is used, the rod or wire can be withdrawn proximally to permit the outer tubular body of the needle to deform from the first configuration into the second configuration under action of the superelastic material 114. In some implementations, the needle delivery catheter can be provided without sheath 420, but including wire 415 and a removable protective tip over the tip 104 of the needle 100 (not shown).

In further accordance with the disclosure, a suturing device is provided to perform a suturing operation with a needle as set forth herein.

For purposes of illustration, and not limitation, FIG. 8 presents a suturing device 500. Device 500 that includes a housing 510 defining a needle track 520 therein to slidably receive a suturing needle (e.g., 100) therein, and a drive 530 to advance the suturing needle along the needle track 520, and across a gap 512 in the housing through a tissue segment (not shown) that is disposed or otherwise pulled into the gap. The drive 530 can be configured to engage and disengage from the suturing needle (e.g., 100) to advance the needle through the tissue segment. The suturing device 500 can further include a needle delivery catheter 400 as described herein, as well as a tissue capture catheter 600 to grasp and manipulate tissue.

An endoscope 700 can be operably coupled to the suturing device 500, which can provide one or more lumens therethrough for slidably receiving the needle delivery catheter 400 and the tissue capture catheter 600. If desired, the endoscope 700 may be oriented obliquely with respect to the suturing needle to provide enhanced visualization of a suturing procedure. This facilitates viewing the passage of the needle across the gap (e.g., 512) using a camera(s) disposed in one (or more) of a plurality of passageways defined along the length of the endoscope 700. Endoscope 700 may be a conventional endoscope, or an endoscope that is specially configured for use with the suturing device. Further conduits may be defined along the endoscope 700 to perform aspiration procedures, to inject a beneficial agent into the region of the suturing site, to provide illumination, and/or to introduce one or more elongate tools into the region where suturing is occurring.

In operation, suturing device 500 receives the sheath 420 down a passageway defined through the endoscope 700 (or through a separate lumen) into a proximal end of the housing 510 along a pathway that is preferably tangential to the needle track 520. Specifically, the needle delivery catheter 400, containing the needle (e.g., 100, 200) can be advanced along a passageway of the endoscope and into the housing 510 of the suturing device 500 toward the needle track 520.

After a distal end portion of the needle delivery catheter 400 has been delivered adjacent to the needle track 520, the needle (e.g., 100) can be pushed distally out of the sheath 420, for example, by pushing distally on wire 115, or on an elongate delivery rod that pushes against the proximal end 102 of the needle 100. As the needle 100 is advanced into the needle track 520, and once the stiffening wire is removed (if provided), the superelastic element 114 within the outer tubular body 110 of the needle 100 will cause the needle 100 to deform into the second, arcuate configuration that is sized to be slidably received within the arcuate needle track 520.

To help advance the needle 100 along the needle track 520, the drive 530 of the suturing device 500 may be actuated to pull the needle 100 fully out of the sheath 420, for example, and to advance the needle across the gap 512 to suture tissue. Once the needle is fully mounted within the needle track 520, a distal end portion of the suturing device 500 can be advanced proximate tissue to be sutured. The suturing needle can then be advanced along the needle track 520 through the tissue a plurality of times to form a plurality of stitches with the trailing suture (e.g., 130). The tissue capture catheter 600 can be advanced along a distal direction and anchored into tissue to be sutured, such as by rotating the catheter 600 to screw the anchor into the tissue. The tissue can then be retracted into a pathway of the needle 100 prior to suturing, such as by pulling a plication of tissue into the gap 512 in the suturing device 500.

In accordance with further aspects, after the stitches have been formed, the needle delivery catheter 400 can be re-engaged with the suturing needle 100 and the needle can be removed from the suturing device by removing the delivery catheter 400. The needle 100 can be pulled proximally into the sheath 420, for example, but pulling proximally on the suturing material 130, which is still attached to the needle 100. If desired, a stabilizing wire can be reintroduced distally along the lumen of sheath 420 and re-engaged with an interior portion of the needle. The combined pulling of the suture and pushing of the sheath and/or stabilizing wire 115 can act to straighten the arcuate needle back toward the first configuration, permitting the needle 100 to be removed from the suturing device 500.

The needle (e.g., 100, 200) can be formed from a variety of materials using a variety of manufacturing processes. For example, the needle can be formed from metallic materials as well as polymeric and/or composite materials. In some implementations, the needle body can be formed from an additive manufacturing process, such as three-dimensional (“3D”) printing. Alternatively, the needle (e.g., 100, 200) can be formed at least on part from a length of hypotube material having a desired cross-sectional shape. The openings (e.g., 116, 116′, 116″, 116′″, 316) can be formed using a material removal process, such as a laser ablation process or chemical etching process, among others. In some implementations, the outer surface of the needle (e.g., 300) can define at least one opening therein (or indentation, as desired). If an indentation is provided, the indentation can be formed by at least one facet, or two or more intersecting facets, or alternative surfaces to interface with a needle advancement drive of a suturing device to advance the needle during a suturing procedure.

Implementations of devices in accordance with the present disclosure can be used to perform an Endoscopic Sleeve Gastroplasty (“ESG”) procedure, wherein a device (e.g., 500) can be deployed into a patient's stomach, and an elongate tissue capture catheter (e.g., 600) can be used to grab portions of the stomach lining to form a plication, and the plication cab then be held in place by passing needle (e.g., 100, 200) through the plication. This procedure can be repeated until all desired plications are formed. Tension can then be applied to the suture (e.g., 130) passing through the plications to reduce the volume of the stomach.

Aspects of this disclosure herein can be part of a computer-assisted teleoperational manipulator system, sometimes referred to as a robotically-assisted manipulator system or a robotic system. The manipulator system can include one or more manipulators that can be operated with the assistance of an electronic controller (e.g., computer) to move and control functions of one or more instruments when coupled to the manipulators.

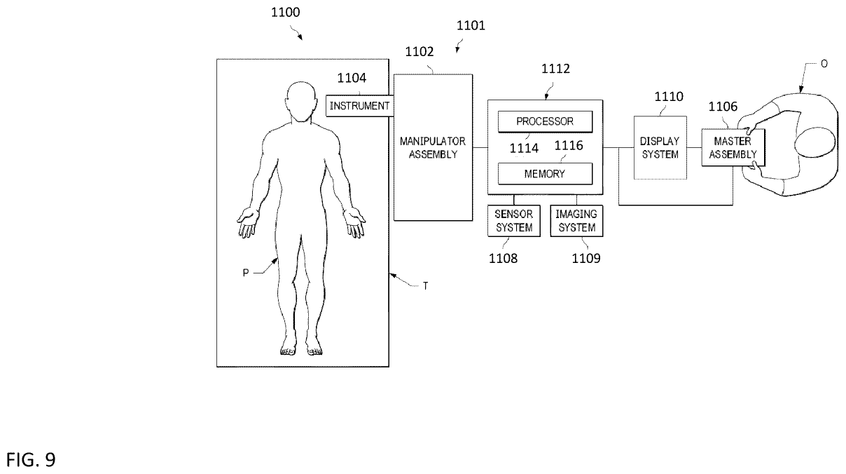

FIG. 9 illustrates an embodiment of a robotically-assisted manipulator system for use with the tools described herein. The manipulator system can be used, for example, in surgical, diagnostic, therapeutic, biopsy, or non-medical procedures, and is generally indicated by the reference numeral 1100. As shown in FIG. 9, a robotically-assisted manipulator system 1100 can include one or more manipulator assemblies 1102 for operating one or more medical instrument systems 1104 in performing various procedures on a patient P positioned on a table T in a medical environment 1101. For example, the manipulator assembly 1102 can drive catheter or end effector motion, can apply treatment to target tissue, and/or can manipulate control members. The manipulator assembly 1102 can be teleoperated, non-teleoperated, or a hybrid teleoperated and non-teleoperated assembly with select degrees of freedom of motion that can be motorized and/or teleoperated and select degrees of freedom of motion that can be non-motorized and/or non-teleoperated. An operator input system 1106, which can be inside or outside of the medical environment 1101, generally includes one or more control devices for controlling manipulator assembly 1102. Manipulator assembly 1102 supports medical instrument system 1104 and can optionally include a plurality of actuators or motors that drive inputs on medical instrument system 1104 in response to commands from a control system 1112. The actuators can optionally include drive systems that when coupled to medical instrument system 1104 can advance medical instrument system 1104 into a naturally or surgically created anatomic orifice. Other drive systems can move the distal end of medical instrument in multiple degrees of freedom, which can include three degrees of linear motion (e.g., linear motion along the X, Y, Z Cartesian axes) and in three degrees of rotational motion (e.g., rotation about the X, Y, Z Cartesian axes). The manipulator assembly 1102 can support various other systems for irrigation, treatment, or other purposes. Such systems can include fluid systems (including, for example, reservoirs, heating/cooling elements, pumps, and valves), generators, lasers, interrogators, and ablation components.

Robotically-assisted manipulator system 1100 also includes a display system 1110 for displaying an image or representation of the surgical site and medical instrument system 1104 generated by an imaging system 1109 which can include an imaging system, such as an endoscopic imaging system. Display system 1110 and operator input system 1106 can be oriented so an operator O can control medical instrument system 1104 and operator input system 1106 with the perception of telepresence. A graphical user interface can be displayable on the display system 1110 and/or a display system of an independent planning workstation.

In some examples, the endoscopic imaging system components of the imaging system 1109 can be integrally or removably coupled to medical instrument system 1104. However, in some examples, a separate imaging device, such as an endoscope, attached to a separate manipulator assembly can be used with medical instrument system 1104 to image the surgical site. The endoscopic imaging system 1109 can be implemented as hardware, firmware, software, or a combination thereof which interact with or are otherwise executed by one or more computer processors, which can include the processors of the control system 1112. It will be appreciated that either an integrated endoscopic device or a separate endoscope can serve as an endoscope that supports the suturing device (e.g., 500) set forth herein.

Robotically-assisted manipulator system 1100 can also include a sensor system 1108. The sensor system 1108 can include a position/location sensor system (e.g., an actuator encoder or an electromagnetic (EM) sensor system) and/or a shape sensor system (e.g., an optical fiber shape sensor) for determining the position, orientation, speed, velocity, pose, and/or shape of the medical instrument system 1104. The sensor system 1108 can also include temperature, pressure, force, or contact sensors or the like.

Robotically-assisted manipulator system 1100 can also include a control system 1112. Control system 1112 includes at least one memory 1116 and at least one computer processor 1114 for effecting control between medical instrument system 1104, operator input system 1106, sensor system 1108, and display system 1110. Control system 1112 also includes programmed instructions (e.g., a non-transitory machine-readable medium storing the instructions) to implement a procedure using the robotically-assisted manipulator system including for navigation, steering, imaging, engagement feature deployment or retraction, applying treatment to target tissue (e.g., via the application of energy), or the like.

Control system 1112 can optionally further include a virtual visualization system to provide navigation assistance to operator O when controlling medical instrument system 1104 during an image-guided surgical procedure. Virtual navigation using the virtual visualization system can be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. The control system 1112 can use a pre-operative image to locate the target tissue (using vision imaging techniques and/or by receiving user input) and create a pre-operative plan, including an optimal first location for performing treatment. The pre-operative plan can include, for example, a planned size to expand an expandable device, a treatment duration, a treatment temperature, and/or multiple deployment locations.

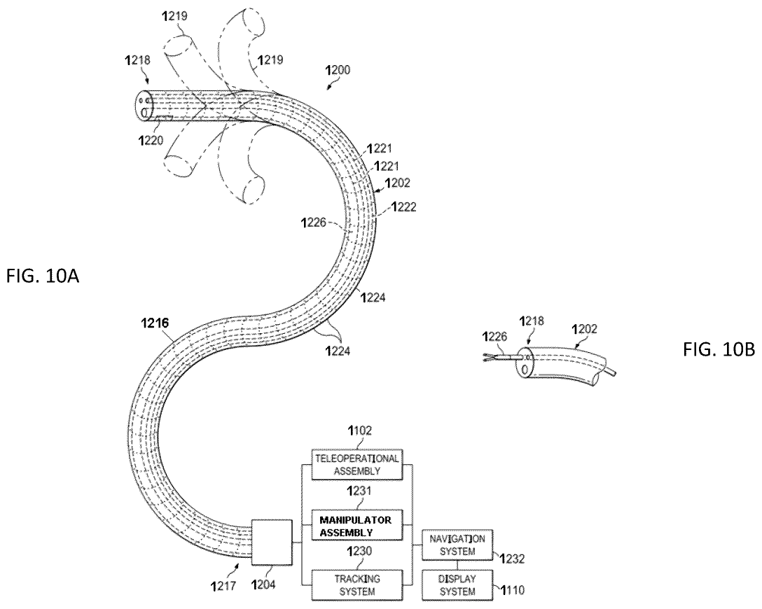

FIG. 10A shows a medical instrument system 1200 according to some embodiments. In some embodiments, medical instrument system 1200 can be used in an image-guided medical procedure. In some examples, medical instrument system 1200 can be used for non-teleoperational exploratory procedures or in procedures involving traditional manually operated medical instruments, such as endoscopy. In some embodiments, medical instrument system 1200 is interchangeable with, or a variation of, medical instrument system 1104 of FIG. 9.

Medical instrument system 1200 includes elongate flexible device 1202, such as a flexible catheter or endoscope (e.g., gastroscope, bronchoscope), coupled to a drive unit 1204. Elongate flexible device 1202 includes a flexible body 1216 having proximal end 1217 and distal end, or tip portion, 1218. In some embodiments, flexible body 1216 has an approximately 14-20 mm outer diameter. Other flexible body outer diameters can be larger or smaller. Flexible body 1216 can have an appropriate length to reach certain portions of the anatomy, such as the lungs, sinuses, throat, or the upper or lower gastrointestional region, when flexible body 1216 is inserted into a patient's oral or nasal cavity. The suturing device (e.g., 500) can be coupled to elongate flexible device 1202 as desired in lieu of endoscope 300.

Medical instrument system 1200 optionally includes a tracking system 1230 for determining the position, orientation, speed, velocity, pose, and/or shape of distal end 1218 and/or of one or more segments 1224 along flexible body 1216 using one or more sensors and/or imaging devices. The entire length of flexible body 1216, between distal end 1218 and proximal end 1217, can be effectively divided into segments 1224. Tracking system 1230 can optionally be implemented as hardware, firmware, software or a combination thereof which interact with or are otherwise executed by one or more computer processors, which can include the processors of control system 1112 in FIG. 9.

Tracking system 1230 can optionally track distal end 1218 and/or one or more of the segments 1224 using a shape sensor 1222. In some embodiments, tracking system 1230 can optionally and/or additionally track distal end 1218 using a position sensor system 1220, such as an electromagnetic (EM) sensor system. In some examples, position sensor system 1220 can be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point.

Flexible body 1216 includes one or more channels 1221 sized and shaped to receive one or more medical instruments 1226. In some embodiments, flexible body 1216 includes two channels 1221 for separate instruments 1226, however, a different number of channels 1221 can be provided. FIG. 9B is a simplified diagram of flexible body 1216 with medical instrument 1226 extended according to some embodiments. In some embodiments, medical instrument 1226 can be used for procedures and aspects of procedures, such as surgery, biopsy, ablation, mapping, imaging, illumination, irrigation, or suction. Medical instrument 1226 can be deployed through channel 1221 of flexible body 1216 and used at a target location within the anatomy. Medical instrument 1226 can include, for example, image capture devices, biopsy instruments, ablation instruments, catheters, laser ablation fibers, and/or other surgical, diagnostic, or therapeutic tools. Medical tools can include end effectors having a single working member such as a scalpel, a blunt blade, a lens, an optical fiber, an electrode, and/or the like. Other end effectors can include, for example, forceps, graspers, balloons, needles, scissors, clip appliers, and/or the like. Other end effectors can further include electrically activated end effectors such as electrosurgical electrodes, transducers, sensors, imaging devices and/or the like.

Medical instrument 1226 can be advanced from the opening of channel 1221 to perform the procedure and then retracted back into the channel when the procedure is complete. Medical instrument 1226 can be removed from proximal end 1217 of flexible body 1216 or from another optional instrument port (not shown) along flexible body 1216. The medical instrument 1226 can be used with an image capture device (e.g., an endoscopic camera) also within the elongate flexible device 1202. Alternatively, the medical instrument 1226 can itself be the image capture device.

Medical instrument 1226 can additionally house cables, linkages, or other actuation controls (not shown) that extend between its proximal and distal ends to controllably the bend distal end of medical instrument 1226. Flexible body 1216 can also house cables, linkages, or other steering controls (not shown) that extend between drive unit 1204 and distal end 1218 to controllably bend distal end 1218 as shown, for example, by broken dashed line depictions 1219 of distal end 1218. In some examples, at least four cables are used to provide independent “up-down” steering to control a pitch motion of distal end 1218 and “left-right” steering to control a yaw motion of distal end 1218. In embodiments in which medical instrument system 1200 is actuated by a robotically-assisted assembly, drive unit 1204 can include drive inputs that removably couple to and receive power from drive elements, such as actuators, of the teleoperational assembly. In some embodiments, medical instrument system 1200 can include gripping features, manual actuators, or other components for manually controlling the motion of medical instrument system 1200. The information from tracking system 1230 can be sent to a navigation system 1232 where it is combined with information from visualization system 1231 and/or the preoperatively obtained models to provide the physician or other operator with real-time position information.

This description and the accompanying drawings that illustrate various embodiments should not be taken as limiting. Various mechanical, compositional, structural, electrical, and operational changes may be made without departing from the scope of this description and the invention as claimed, including equivalents. In some instances, well-known structures and techniques have not been shown or described in detail so as not to obscure the disclosure. Like numbers in two or more figures represent the same or similar elements. Furthermore, elements and their associated features that are described in detail with reference to one embodiment may, whenever practical, be included in other embodiments in which they are not specifically shown or described. For example, if an element is described in detail with reference to one embodiment and is not described with reference to another embodiment, the element may nevertheless be claimed as included in the other embodiment.

For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Further, this description's terminology is not intended to limit the invention. For example, spatially relative terms—such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the illustrative term “below” can encompass both positions and orientations of above and below. A device may be otherwise oriented (rotated 90 degrees or at other orientations) and the spatially relative descriptors used herein interpreted accordingly.

Further modifications and alternative embodiments will be apparent to those of ordinary skill in the art in view of the disclosure herein. For example, the devices and methods may include additional components or steps that were omitted from the diagrams and description for clarity of operation. Accordingly, this description is to be construed as illustrative only and is for the purpose of teaching those skilled in the art the general manner of carrying out the present teachings. It is to be understood that the various embodiments shown and described herein are to be taken as illustrative. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. Changes may be made in the elements described herein without departing from the spirit and scope of the present teachings and following claims.

It is to be understood that the particular examples and embodiments set forth herein are non-limiting, and modifications to structure, dimensions, materials, and methodologies may be made without departing from the scope of the present teachings.

Other embodiments in accordance with the present disclosure will be apparent to those skilled in the art from consideration of the specification and practice of the invention disclosed herein. It is intended that the specification and examples be considered as illustrative and for example only, with the following claims being entitled to their fullest breadth, including equivalents, under the applicable law.

1. A suturing needle comprising a needle body having a leading end and a trailing end, the needle body comprising an outer tubular body having a proximal end and a distal end configured to pierce tissue, and at least one at least one elongate inner body made of superelastic material disposed within and operably coupled to the outer tubular body, wherein the outer tubular body deforms from a first configuration into a second configuration in response to the application of force by the at least one elongate inner body on the outer tubular body.

2. The suturing needle of claim 1, wherein the outer tubular body defines at least one opening therethrough when the outer tubular body is in the first configuration that decreases in spatial extent when the outer tubular body deforms from the first configuration into the second configuration.

3. The suturing needle of claim 1, wherein the at least one opening comprises a plurality of openings defined along a length of the outer tubular body.

4. The suturing needle of claim 3, wherein the first configuration is a generally linear configuration.

5. The suturing needle of claim 3, wherein the second configuration is a curved configuration.

6. The suturing needle of claim 5, wherein the second configuration is an arcuate configuration.

7. The suturing needle of claim 3, wherein at least one of the first configuration and the second configuration defines a splined shape.

8. The suturing needle of claim 3, wherein at least one of the first configuration and the second configuration defines an out of plane shape.

9. The suturing needle of claim 3, wherein each opening in the plurality of openings is defined along a first lateral side of the outer tubular body.

10. The suturing needle of claim 9, wherein no openings are defined along a second lateral side of the outer tubular body opposite the first lateral side of the outer tubular body.

11. The suturing needle of claim 9, wherein a second lateral side of the outer tubular body opposite the first lateral side of the outer tubular body is defined by a continuous surface free from openings.

12. The suturing needle of claim 1, wherein the outer tubular body is not diametrically expandable.

13. The suturing needle of claim 1, wherein the outer tubular body is not diametrically contractable.

14. The suturing needle of claim 1, wherein a cross-sectional height of the outer tubular body does not change when the outer tubular body deforms from the first configuration into the second configuration.

15. The suturing needle of claim 1, wherein the outer tubular body has sufficient column strength when in the second configuration to be advanced through tissue to suture the tissue without deformation of the suturing needle.

16. The suturing needle of claim 15, wherein the outer tubular body is generally toroidally shaped when in the second configuration.

17. The suturing needle of claim 15, wherein the outer tubular body resists deformation when advanced through tissue in the second configuration during a suturing operation.

18-51. (canceled)

52. A suturing needle comprising a needle body having a leading end and a trailing end, the needle body comprising a first body having a proximal end and a distal end configured to pierce tissue, and at least one at least one elongate body made of superelastic material operably coupled to the first body, wherein the first body deforms from a first configuration into a second configuration in response to the application of force by the at least one elongate body on the first body.

53. The suturing needle of claim 52, wherein the at least one elongate body is disposed parallel to the first body.

54. The suturing needle of claim 52, wherein the least one elongate body is operably coupled to the first body at a plurality of locations along a length of the first body.
